(19) United States
(12) Patent Application Publication
(10) Pub. No.: US 2025/0255804 A1
(43) Pub. Date: Aug. 14, 2025

(54) LIQUID INJECTABLE COMPOSITIONS OF LURBINECTEDIN

(71) Applicant: DR. REDDY’S LABORATORIES LIMITED, Hyderabad (IN)

(72) Inventors: Mukesh BOTHRA, Secunderabad (IN); Sathish Kumar PASUMARTHI, Hyderabad (IN); Naresh PUSUNURU, Hyderabad (IN)

(21) Appl. No.: 19/049,310

(22) Filed: Feb. 10, 2025

(30) Foreign Application Priority Data
Feb. 10, 2024 (IN) 202341053567

(51) Int. Cl. A61K 9/00; A61K 9/08; A61K 31/4995; A61K 47/02; A61K 47/10; A61K 47/40

(52) U.S. Cl. CPC A61K 9/0019; A61K 9/08; A61K 31/4995; A61K 47/02; A61K 47/10; A61K 47/40

(57) ABSTRACT

The present invention relates to liquid injectable compositions of lurbinectedin or pharmaceutical acceptable salts, hydrates, solvates, polymorphs, and mixtures thereof. More particularly, the present invention relates to liquid injectable compositions of lurbinectedin which are ready-to-dilute comprising of suitable solvent system.

### CROSS REFERENCE TO RELATED APPLICATION

This application takes priority from an Indian application number IN 202341053567, filed on Feb. 10, 2024, which is hereby incorporated by reference in its entirety.

### FIELD OF INVENTION

The present invention relates to liquid injectable compositions of lurbinectedin or pharmaceutical acceptable salts, hydrates, solvates, polymorphs, and mixtures thereof.

### BACKGROUND OF THE INVENTION

Lurbinectedin is alkylating drug, approved and indicated for the treatment of adult patients with metastatic small cell lung cancer (SCLC) with disease progression on or after platinum-based chemotherapy. Lurbinectedin is structurally represented as

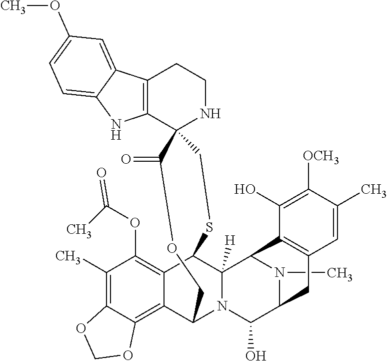

Lurbinectedin is available in the US market with the Brand Name Zepzelca® and marketed by Jazz Pharmaceuticals as an injection, supplied as a lyophilized powder in a single-dose vial for reconstitution for intravenous use. U.S. Pat. No. 7,763,615 describes lurbinectedin and related compounds.

Each single dose vial of Zepzelca® contains the 4 mg of lurbinectedin and the following inactive ingredients: sucrose (800 mg), lactic acid (22.1 mg), and sodium hydroxide (5.1 mg). Before use, the lyophilizate is reconstituted by addition of 8 mL Sterile Water for Injection USP, yielding a solution containing 0.5 mg/mL lurbinectedin. The lyophilized composition should be stored at 2° to 8° C. (36° to 46° F.).

WO2021228414A1 and WO2021098992A1 disclose lyophilized composition of lurbinectedin for intravenous use, comprising 4 mg lurbinectedin; an organic carboxylic acid; and a disaccharide; wherein the lyophilized composition is formulated such that reconstitution with 8 mL of water will yield a solution having a pH of 3.5 to 4.5. These references disclose a disaccharide include sucrose, trehalose or lactose, or a combination thereof and a buffer derived from an organic carboxylic acid include lactic acid, butyric acid, propionic acid, acetic acid, succinic acid, citric acid, ascorbic acid, tartaric acid, malic acid, maleic acid, fumaric acid, and glutamic acid, aspartic acid, gluconic acid, a-ketoglutaric, particularly, lactic acid or succinic acid. These references further disclose a lyophilized composition comprising 4 mg lurbinectedin; 22.1 mg lactic acid; 5.1 mg sodium hydroxide; and 800 mg sucrose. These references also disclose that lurbinectedin has limited aqueous solubility and it was found that solubility is improved in the bulk solution by first forming a concentrated pre-solution of the lurbinectedin in a buffer derived from an organic acid, for example lactic acid, succinic acid, citric acid, or acetic acid which is further diluted with water for injection. Additionally, as per the regulatory document of Zepzelca® available from US FDA website, lurbinectedin is insoluble or practically insoluble in water but solubility increases at acidic pH.

WO2021099635A1 discloses polymorphic form B of lurbinectedin free base and manufacturing process thereof. It further discloses use of Form B in preparing a bulk solution for lyophilizing by dissolving form B of lurbinectedin in an acidic medium, mixing the pre-dissolved lurbinectedin with the other components of the bulking solution. The process further comprises freeze-drying the bulk solution.

The present labeling requirement of the marketed formulation provides instructions for the re-constituted/diluted solution to be used immediately and if not used immediately, it can only be stored prior to administration at either room temperature/ambient light or 2° C.-8° C. (36° F.-46° F.) for up to 24 hours (including infusion time) following reconstitution.

Further, based on the body surface area (BSA), appropriate amount of reconstituted solution is diluted into 100 ml (if administered through central venous line)/250 ml (if administered through peripheral venous line) of parenterally acceptable diluents such as 0.9% saline (normal saline) or 5% Dextrose Injection USP through central or peripheral venous line. This involves multiple steps dilution, reconstitution, and further dilution. During reconstitution there are chances that the nursing personnel may get occupational harmful exposure to drug. To avoid adverse effects of antineoplastic agents due to such exposure of anticancer drugs, it is recommended that these substances be handled with great care, contact with skin and eyes avoided and they should not be inhaled. Additionally, reconstitution introduces the potential for dilution errors and in some cases a longer shaking period is required for solubilizing the drug completely. Parenteral formulations provided in the ready-to-dilute form doesn't require the step of reconstitution consequently, will be helpful in avoiding unnecessary exposure and dilution errors.

Accordingly, there exists a need to develop stable liquid, ready-to-dilute or ready-to-use compositions of lurbinectedin or pharmaceutical acceptable salts or solvates thereof, for ease of administration and to prevent unnecessary exposure to healthcare professionals as it is a hazardous drug.

### SUMMARY OF INVENTION

The present invention relates to liquid injectable compositions comprising lurbinectedin or its pharmaceutically acceptable salts, hydrates, solvates, polymorphs, and mixtures thereof and solvent system suitable for injection.

The present invention relates to liquid injectable compositions comprising lurbinectedin or its pharmaceutically acceptable salts, hydrates, solvates, polymorphs, and mixtures thereof and solvent system suitable for injection, wherein the liquid injectable compositions are ready-to-dilute.

The present invention relates to liquid injectable compositions comprising lurbinectedin or its pharmaceutically acceptable salts, hydrates, solvates, polymorphs, and mixtures thereof and solvent system suitable for injection, wherein the liquid injectable compositions are ready-to-use.

Another embodiment of the present invention relates to liquid injectable compositions comprising from about 0.01 mg/mL to about 2.0 mg/mL of Lurbinectedin or its pharmaceutically acceptable salts, hydrates, solvates, polymorphs, and mixtures thereof; and solvent system suitable for injection, wherein the liquid injectable compositions are ready-to-dilute.

Another embodiment of the present invention relates to liquid injectable compositions comprising from about 0.01 mg/mL to about 2.0 mg/mL of Lurbinectedin or its pharmaceutically acceptable salts, hydrates, solvates, polymorphs, and mixtures thereof; and solvent system suitable for injection, wherein the liquid injectable compositions are ready-to-use.

Another embodiment of the present invention relates to liquid injectable compositions comprising lurbinectedin or its pharmaceutically acceptable salts, hydrates, solvates, polymorphs, and mixtures thereof and solvent system suitable for injection, wherein the liquid injectable compositions are ready-to-dilute, wherein the solvent system comprises about 1% v/v to about 100% v/v of solvent(s), preferably from about 5% v/v to about 95% v/v of solvent(s) and most preferably from about 10% v/v to about 90% v/v of solvent(s).

Another embodiment of the present invention relates to liquid injectable compositions comprising lurbinectedin or its pharmaceutically acceptable salts, hydrates, solvates, polymorphs, and mixtures thereof and solvent system suitable for injection, wherein the liquid injectable compositions are ready-to-use, wherein the solvent system comprises about 1% v/v to about 100% v/v of solvent(s), preferably from about 5% v/v to about 95% v/v of solvent(s), and most preferably from about 10% v/v to about 90% v/v of solvent(s).

Another embodiment of the present invention relates to liquid injectable compositions comprising lurbinectedin or its pharmaceutically acceptable salts, hydrates, solvates, polymorphs, and mixtures thereof; solvent system suitable for injection and a solubilizer.

Another embodiment of the present invention relates to liquid injectable compositions comprising lurbinectedin or its pharmaceutically acceptable salts, hydrates, solvates, polymorphs, and mixtures thereof; solvent system suitable for injection and about 1 mg/mL to about 1000 mg/mL solubilizer.

Another embodiment of the present invention relates to liquid injectable compositions comprising lurbinectedin or its pharmaceutically acceptable salts, hydrates, solvates, polymorphs, and mixtures thereof; solvent system suitable for injection, about 1 mg/mL to about 1000 mg/mL solubilizer and a stabilizer.

Another embodiment of the present invention relates to liquid injectable compositions comprising lurbinectedin or its pharmaceutically acceptable salts, hydrates, solvates, polymorphs, and mixtures thereof; solvent system suitable for injection, about 1 mg/mL to about 1000 mg/mL solubilizer and about 5 mg/mL to about 100 mg/mL stabilizer.

Another embodiment of the present invention relates to liquid injectable compositions comprising lurbinectedin or its pharmaceutically acceptable salts, hydrates, solvates, polymorphs, and mixtures thereof; solvent system suitable for injection, about 1 mg/mL to about 1000 mg/mL solubilizer, about 5 mg/mL to about 100 mg/mL stabilizer and a buffering agent.

Another embodiment of the present invention relates to liquid injectable compositions comprising lurbinectedin or its pharmaceutically acceptable salts, hydrates, solvates, polymorphs, and mixtures thereof; solvent system suitable for injection, about 1 mg/mL to about 1000 mg/mL solubilizer, about 5 mg/mL to about 100 mg/mL stabilizer, about 0.01 mg/mL to about 100 mg/mL buffering agent, and wherein the composition has a pH of about 1 to about 14.

Another embodiment of the present invention relates to liquid injectable compositions comprising lurbinectedin or its pharmaceutically acceptable salts, hydrates, solvates, polymorphs, and mixtures thereof; solvent system suitable for injection, about 1 mg/mL to about 1500 mg/mL solubilizer, about 5 mg/mL to about 100 mg/mL stabilizer, about 0.01 mg/mL to about 100 mg/mL buffering agent, about 0.0001 mg/mL to about 10 mg/mL antioxidants.

The present invention relates to ready-to-dilute liquid pharmaceutical composition comprising:
a) 0.1 mg/mL to 2 mg/mL of lurbinectedin or a pharmaceutically acceptable salt thereof,
b) polyethylene glycol,
c) cyclodextrin, and
d) optionally, one or more other pharmaceutical excipients,
   wherein the composition has a pH between 2.0 to 6.0.

The present invention relates to a liquid pharmaceutical composition comprising:
a) 0.1 mg/mL to 2 mg/mL of lurbinectedin or a pharmaceutically acceptable salt thereof,
b) about 100 mg/mL to about 500 mg/mL of polyethylene glycol,
c) about 5 mg/mL to about 50 mg/mL of cyclodextrin, and
d) optionally, one or more other pharmaceutical excipients,
   wherein the composition has a pH between 2.0 to 6.0.

The present invention relates to a liquid pharmaceutical composition comprising:
a) 0.1 mg/mL to 1 mg/mL of lurbinectedin or a pharmaceutically acceptable salt thereof,
b) about 200 mg/mL to about 300 mg/mL of polyethylene glycol,
c) about 10 mg/mL to about 30 mg/mL of sulfobutylether-β-cyclodextrin,
d) a co-solvent selected from the group consisting of benzyl alcohol or ethanol, and
e) hydrochloric acid,
   wherein the composition has a pH between 2.0 to 6.0.

The present invention relates to a liquid pharmaceutical composition comprising:
a) 0.1 mg/mL to 2 mg/mL of lurbinectedin or a pharmaceutically acceptable salt thereof,
b) about 100 mg/mL to about 500 mg/mL of polyethylene glycol,
c) about 5 mg/mL to about 50 mg/mL of cyclodextrin, and
d) optionally, one or more other pharmaceutical excipients,
   wherein the composition maintains at least 98% of Lurbinectedin content and/or contains less than 2% of total impurities as determined by HPLC, after storage at 2-8° C. for a period of at least 6 months.

### DETAILED DESCRIPTION OF THE INVENTION

The present invention relates to liquid injectable compositions comprising lurbinectedin or its pharmaceutically acceptable salts, hydrates, solvates, polymorphs, and mixtures thereof and solvent system suitable for injection.

The present invention relates to liquid injectable compositions comprising lurbinectedin or its pharmaceutically acceptable salts, hydrates, solvates, polymorphs, and mixtures thereof and solvent system suitable for injection, wherein the liquid compositions are re-constituted from lyophilized forms of lurbinectedin.

The present invention relates to liquid injectable compositions comprising lurbinectedin or its pharmaceutically acceptable salts, hydrates, solvates, polymorphs, and mixtures thereof and solvent system suitable for injection, wherein the liquid injectable compositions are ready-to-dilute.

The present invention relates to liquid injectable compositions comprising lurbinectedin or its pharmaceutically acceptable salts, hydrates, solvates, polymorphs, and mixtures thereof and solvent system suitable for injection, wherein the liquid injectable compositions are ready-to-use.

As used herein the term “liquid” refers to formulation, which is ready to use, ready to dilute or compositions reconstituted from lyophilized formulation of lurbinectedin.

As used herein the term “ready-to-dilute” refers to a formulation which can be directly combined with a parenterally acceptable diluent (e.g., dextrose solution, saline solution, or any other infusion medium) and then administered to a patient. A sufficient amount of a concentrated, ready to dilute liquid formulation of lurbinectedin admixed with a suitable fixed volume diluent container such as a bag containing 50, 100, 250 ml of 0.9% sodium chloride injection or dextrose Injection.

As used herein the term “ready-to-use” refers to any preparation of lurbinectedin which can be administered to patient directly without any further dilution or processing.

As used herein, the terms “composition” and “formulation” refer to preparations comprising lurbinectedin or its pharmaceutically acceptable salts, hydrates, solvates, polymorphs, and mixtures thereof in a form suitable for administration to a human of a mammal.

The term “solvent” refers to an ingredient used for dissolving an ingredient.

As used herein, the term “stable” refers to physical and chemical stability for commercially significant periods, such as at least 1 week or 1 month or 3 months or 6 months or 1 year or 2 years or 3 years, without significant physical stability (description, clarity etc.) and chemical degradation. Stable or stability may represent stability when stored at 2° C.-8° C. or at ambient conditions (e.g., 25° C.) or elevated temperatures (e.g., 40° C.).

According to another embodiment, the present invention relates to the liquid injectable composition comprising from about 0.01 mg/mL to about 2.0 mg/mL of lurbinectedin or its pharmaceutically acceptable salts, hydrates, solvates, polymorphs, and mixtures thereof; preferably from about 0.01 mg/mL to about 0.8 mg/mL of lurbinectedin or its pharmaceutically acceptable salts, hydrates, solvates, polymorphs, and mixtures thereof.

The “solvent system” refers to mixture of one or more solvents in a definite proportion which is suitable for dissolving the active ingredient. The solvent system in the present invention includes aqueous solvents, non-aqueous solvents or mixtures thereof. The aqueous solvents may include but not limited to purified water, dextrose solution, saline solution. The non-aqueous solvents are known in the art and include but not limited to alkyl alcohols, for example ethanol, benzyl alcohol; alkyl glycols for example, ethylene glycol, propylene glycol; and butylene glycol; glycerol; polysorbates for examples TWEEN 20, TWEEN 40 and TWEEN 80; benzyl benzoate; polyalkylene glycols, such as polyethylene glycol, polypropylene glycol, and polybutylene glycol, dimethyl acetamide, dimethyl sulfoxide, acetone, tetrahydrofuran, 1, 4-dioxone, acetonitrile, dimethyl formamide, propylene carbonate, 1-methyl-2-pyrrolidione, 1, 3-dimethyl-2-imidazolidinone or mixtures thereof. Preferably, the mixtures of polyalkylene glycols such as PEG 300, PEG 400 along with non-ionic liquid polymer of the alkyl aryl polyether alcohol type such as Tyloxapol and other solvents such as ethanol, glycerol, castor oil, polyethoxylated castor oil etc.

Another embodiment of the present invention relates to liquid injectable compositions comprising lurbinectedin or its pharmaceutically acceptable salts, hydrates, solvates, polymorphs, and mixtures thereof and solvent system suitable for injection, wherein the liquid injectable compositions are ready-to-dilute, wherein the solvent system comprises about 1% v/v to about 100% v/v of solvent(s), preferably from about 5% v/v to about 95% v/v of solvent(s) and most preferably from about 10% v/v to about 90% v/v of solvent(s).

Another embodiment of the present invention relates to liquid injectable compositions comprising lurbinectedin or its pharmaceutically acceptable salts, hydrates, solvates, polymorphs, and mixtures thereof and solvent system suitable for injection, wherein the solvent system comprises about 1% v/v to about 100% v/v of solvent, preferably from about 5% v/v to about 95% v/v of solvent and most preferably from about 10% v/v to about 90% v/v of solvent.

Another embodiment of present invention relates to liquid injectable compositions comprising lurbinectedin or its pharmaceutically acceptable salts, hydrates, solvates, polymorphs, and mixtures thereof; solvent system suitable for injection and a solubilizer.

Another embodiment of the present invention relates to liquid injectable compositions comprising lurbinectedin or its pharmaceutically acceptable salts, hydrates, solvates, polymorphs, and mixtures thereof; solvent system suitable for injection, a solubilizer.

The term “solubilizer” refers to any substance which enhances the solubility of the drug in the solvents. Solubilizers in the present invention include but not limited to glycerin, lecithin, castor oil, derivatives of castor oil such as PEG-40 castor oil, polyethylene glycol, polysorbate, sorbitan monopalmitate, povidone, tromethamine, cyclodextrins such as beta cyclodextrin etc.

Another embodiment of the present invention relates to liquid injectable compositions comprising lurbinectedin or its pharmaceutically acceptable salts, hydrates, solvates, polymorphs, and mixtures thereof; solvent system suitable for injection and a solubilizer, wherein the solubilizer is present in an amount of about 1 mg/mL to about 1500 mg/mL. The quantity varies depending on the nature of the solubilizer used.

According to another embodiment, the present invention further comprises stabilizers.

The term “stabilizer” identifies an agent which improves the composition physical and chemical stability for commercially significant periods, such as at least 1 week, 1 month, 2 months, 3 months, 6 months, 1 year, 2 years, 3 years, without significant physical instability (description, clarity etc.) and chemical degradation. Stable or stability may represent stability when stored at 2° C.-8° C. or at ambient conditions (e.g. 25° C.) or elevated temperatures (e.g. 40° C.). Suitable stabilizers include but are not limited to cyclodextrin include hydroxypropyl beta-cyclodextrin (HPBCD) and sulfobutylether beta-cyclodextrin (SBECD), ethylenediaminetetraacetic acid (EDTA), monothioglycerol, glutathione, ascorbic acid, ethylgallate, cysteine etc. The stabilizer is present in the amount of about 5 mg/mL to about 100 mg/mL.

It was found that addition of cyclodextrin to the liquid composition of lurbinectedin decreases the impurities of the composition upon storage at 2-8° C. temperature for at least 1 month or at least 2 months or at least 3 months or at least 5 months.

According to another embodiment, the present invention further comprises buffering agents.

A primary source of pH control can be buffer. Typically, a buffer is present as an acid or a base and its conjugate base or acid, respectively. Suitable examples of buffering agent may include but are not limited to phosphate, Tromethamine (Tris), sodium bicarbonate, sodium carbonate, hydrochloric acid, sodium hydroxide, glycine and mixtures thereof. In certain embodiments the buffering agent may be present in an amount of about 0.01 mg/mL to about 100 mg/mL. The amount of buffering agent used differs based on the buffering agent used in the composition.

The term “carboxylic acid” as used herein the invention refers to an organic acid that contains a carboxyl group (—C(═O)—OH). The carboxylic acid herein includes mono-cocarboxylic acids, di-carboxylic acids, tri-carboxylic acids. The examples of carboxylic acids include lactic acid, acetic acid, butyric acid, propionic acid, acetic acid, succinic acid, citric acid, ascorbic acid, tartaric acid, malic acid, maleic acid, fumaric acid, and glutamic acid, aspartic acid, gluconic acid.

The term “monosaccharides” as used herein the invention refers simple sugars and are the simplest form of carbohydrates that cannot be further broken down into smaller units. The monosaccharides herein include glucose, dextrose, fructose, galactose, or galactose or a combination thereof.

The term “disaccharides” as used herein the invention refers organic compounds containing sugars. The disaccharides herein include sucrose, trehalose or lactose, or a combination thereof.

According to another embodiment, the present invention further comprises antioxidants.

The antioxidants used in the present invention are selected from ascorbic acid, butylated hydroxy toluene (BHT), butylated hydroxy anisole (BHA), methionine, Pentetic acid, sodium sulfite, sodium bisulfite, tocopherol, monothioglycerol, thymol, sodium formaldehyde sulfoxylate, propyl gallate, sodium ascorbate, sodium thiosulfate, sulfur dioxide, Vitamin E Polyethylene Glycol Succinate, potassium metabisulfite and sodium metabisulfite. The amount of the antioxidant present in the composition is preferably from about 0.0001 mg/mL to about 100 mg/mL. The amount of antioxidant used in the composition varies depending on the antioxidant used.

According to another embodiment, the present invention relates to liquid injectable compositions comprising lurbinectedin or its pharmaceutically acceptable salts, hydrates, solvates, polymorphs, and mixtures thereof; solvent system suitable for injection, about 1 mg/mL to about 1500 mg/mL solubilizer, about 5 mg/mL to about 100 mg/mL stabilizer, about 0.01 mg/mL to about 100 mg/mL buffering agent, about 0.0001 mg/mL to about 100 mg/mL antioxidants.

According to another embodiment, the present invention relates to liquid injectable compositions comprising lurbinectedin or its pharmaceutically acceptable salts, hydrates, solvates, polymorphs, and mixtures thereof; solvent system suitable for injection about 1 mg/mL to about 1500 mg/mL solubilizer, about 5 mg/mL to about 100 mg/mL stabilizer, about 0.01 mg/mL to about 100 mg/mL buffering agent, about 0.0001 mg/mL to about 100 mg/mL antioxidants, wherein the liquid injectable compositions are ready-to-dilute.

According to another embodiment, the present invention relates to liquid injectable compositions comprising lurbinectedin or its pharmaceutically acceptable salts, hydrates, solvates, polymorphs, and mixtures thereof; solvent system suitable for injection about 1 mg/mL to about 1500 mg/mL solubilizer, about 5 mg/mL to about 100 mg/mL stabilizer, about 0.01 mg/mL to about 100 mg/mL buffering agent, about 0.0001 mg/mL to about 100 mg/mL antioxidants, wherein the liquid injectable compositions are ready-to-use.

The liquid injectable compositions of lurbinectedin may optionally include one or more pharmaceutically acceptable excipients. The pharmaceutically acceptable excipients may include any one or more of: one or more antibacterial preservatives, including one or more of phenyl mercuric nitrate, thiomersal, benzalkonium chloride, benzethonium chloride, phenol, cresol and chlorobutanol; and tonicity contributors including one or more of sodium chloride, potassium chloride, and alkaline substances including one or more of sodium hydroxide, potassium hydroxide, sodium carbonate and meglumine and salts such as sodium chloride. Additionally, may include one or more chelating agents such as EDTA, tartaric acid etc.

The liquid injectable formulations of the present invention have sufficient stability to have utility as a pharmaceutical agent. Preferably, the formulation has both physical and chemical stability for commercially significant periods, such as at least 1 week, 1 month, 2 months, 3 months, 6 months, 1 year, or 2 years or 3 years, without significant physical instability (description, clarity etc.) and chemical degradation. Stable or stability may represent stability when stored at 2° C.-8° C. or at ambient conditions (e.g. 25° C.) or elevated temperatures (e.g. 40° C.). Percent degradation may be determined by analyzing for impurities by suitable analytical method such as HPLC.

In one embodiment, the present invention relates to liquid pharmaceutical composition comprising:
a) 0.1 mg/mL to 2 mg/mL of lurbinectedin or a pharmaceutically acceptable salt thereof,
b) polyethylene glycol,
c) cyclodextrin, and
d) optionally, one or more other pharmaceutical excipients,
   wherein the composition has a pH between 2.0 to 6.0.

In some embodiments, the liquid pharmaceutical composition described herein is a ready-to-dilute composition.

In one embodiment, the present invention relates to ready-to-dilute liquid pharmaceutical composition comprising:
a) 0.1 mg/mL to 2 mg/mL of lurbinectedin or a pharmaceutically acceptable salt thereof,
b) polyethylene glycol,
c) cyclodextrin, and
d) optionally, one or more other pharmaceutical excipients,
wherein the composition has a pH between 2.0 to 6.0

In one embodiment, the present invention relates to a liquid pharmaceutical composition comprising:
a) 0.1 mg/mL to 2 mg/mL of lurbinectedin or a pharmaceutically acceptable salt thereof,
b) about 100 mg/mL to about 500 mg/mL of polyethylene glycol,
c) about 5 mg/mL to about 50 mg/mL of cyclodextrin, and
d) optionally, one or more other pharmaceutical excipients,
   wherein the composition has a pH between 2.0 to 6.0.

In one embodiment, the present invention relates to a ready-to-dilute liquid pharmaceutical composition comprising:
a) 0.1 mg/mL to 2 mg/mL of lurbinectedin or a pharmaceutically acceptable salt thereof,
b) about 100 mg/mL to about 500 mg/mL of polyethylene glycol,
c) about 5 mg/mL to about 50 mg/mL of cyclodextrin, and
d) optionally, one or more other pharmaceutical excipients,
   wherein the composition has a pH between 2.0 to 6.0.

In some embodiments, the pharmaceutical compositions described herein comprises Lurbinectedin at a concentration of about 0.1 mg/mL to about 1.5 mg/mL or about 0.5 mg/mL to about 1.0 mg/mL. In specific embodiments, the pharmaceutical compositions described herein comprises Lurbinectedin at a concentration of about 0.5 mg/mL.

In some embodiments, the pharmaceutical compositions described herein comprises polyethylene glycol of various molecular weights, for example PEG 300 and PEG 400.

In some embodiments, the pharmaceutical compositions described herein comprises polyethylene glycol at a concentration of about 100 mg/mL to about 450 mg/mL or about 100 mg/mL to about 400 mg/mL or about 100 mg/mL to about 300 mg/mL or about 100 mg/mL to about 200 mg/mL. In specific embodiments, the pharmaceutical compositions described herein comprises polyethylene glycol at a concentration of about 225 mg/mL or about 367 mg/mL.

In some embodiments, the pharmaceutical compositions described herein comprises cyclodextrin such as beta-cyclodextrin. In certain embodiments, the pharmaceutical compositions described herein comprises cyclodextrin such as sulfobutylether β-cyclodextrin (SBECD) or hydroxypropyl β-cyclodextrin.

In certain embodiments, the pharmaceutical compositions described herein comprises cylcodextrin at a concentration of about 5 mg/mL to about 50 mg/mL or about 5 mg/mL to about 40 mg/mL or about 10 mg/mL to about 40 mg/mL or about 10 mg/mL to about 30 mg/mL. In specific embodiments, the pharmaceutical compositions described herein comprises cylcodextrin at a concentration of about 20 mg/mL.

In certain embodiments, the pharmaceutical compositions described herein further comprises a co-solvent. As used herein, the term “co-solvent” refers to a second solvent included in a composition. Exemplary cosolvents include, but are not limited to, dehydrated alcohol, benzyl alcohol, butanediol, isopropanol, alkyl glycols for example, ethylene glycol, propylene glycol, and butylene glycol, glycerol, polysorbate or a mixture of one or more thereof. In certain embodiments, the cosolvent is selected from ethanol, benzyl alcohol or mixtures thereof.

In some embodiments, the pharmaceutical compositions described herein comprises a co-solvent at a concentration of about 1 mg/mL to about 300 mg/mL or 5 mg/mL to about 250 mg/mL or 10 mg/mL to about 100 mg/mL or 20 mg/mL to about 50 mg/mL.

In certain embodiments, the pharmaceutical compositions described herein further comprise a stabilize selected from the group consisting of ethylenediaminetetraacetic acid (EDTA), monothioglycerol, glutathione, ethylgallate, cysteine or mixtures thereof.

In certain embodiments, the pharmaceutical compositions described herein further comprise water for injection.

In some embodiments, the pharmaceutical compositions described herein comprises a water at a concentration of about 100 mg/mL to about 900 mg/mL or 300 mg/mL to about 800 mg/mL or 500 mg/mL to about 700 mg/mL.

In certain embodiments, the pharmaceutical compositions described herein further comprise an inorganic acid. Exemplary inorganic acid include, but are not limited to, hydrochloric acid, sulphuric acid, nitric acid, perchloric acid. In certain embodiments, the inorganic is hydrochloric acid.

The pH of the liquid pharmaceutical compositions described herein is in the range of about 2.0 to about 6.0. In some embodiments, the pH of the liquid pharmaceutical compositions herein is the range of about 2.0 to about 5.0. In some embodiments, the pH of the liquid pharmaceutical compositions described herein is the range of about 2.0 to about 4.0. In some embodiments, the pH of the liquid pharmaceutical compositions described herein is the range of about 3.0 to about 4.0. In some embodiments, the pH of the liquid pharmaceutical compositions described herein is about 4.0, about 4.1, about 4.2, about 4.3, about 4.4, or about 4.5.

In one embodiment, the present invention relates to a ready-to-dilute liquid pharmaceutical composition comprising:
a) 0.1 mg/mL to 1 mg/mL of lurbinectedin or a pharmaceutically acceptable salt thereof,
b) about 100 mg/mL to about 500 mg/mL of polyethylene glycol,
c) about 5 mg/mL to about 50 mg/mL of cyclodextrin,
d) a co-solvent selected from the group consisting of ethanol, benzyl alcohol, butanediol, isopropanol, alkyl glycols for example, ethylene glycol, propylene glycol, and butylene glycol, glycerol, polysorbate or mixtures thereof, and
e) optionally, one or more other pharmaceutical excipients,
   wherein the composition has a pH between 2.0 to 6.0.

In another embodiment, the present invention relates to a ready-to-dilute liquid pharmaceutical composition comprising:
a) 0.1 mg/mL to 1 mg/mL of lurbinectedin or a pharmaceutically acceptable salt thereof,
b) about 100 mg/mL to about 500 mg/mL of polyethylene glycol,
c) about 5 mg/mL to about 50 mg/mL of cyclodextrin,
d) a co-solvent selected from the group consisting of ethanol, benzyl alcohol, butanediol, isopropanol, alkyl glycols for example, ethylene glycol, propylene glycol, and butylene glycol, glycerol, polysorbate or mixtures thereof; and
e) hydrochloric acid;
   wherein the composition has a pH between 2.0 to 6.0.

In another embodiment, the present invention relates to a ready-to-dilute liquid pharmaceutical composition comprising:
a) 0.1 mg/mL to 1 mg/mL of lurbinectedin or a pharmaceutically acceptable salt thereof,
b) about 100 mg/mL to about 500 mg/mL of polyethylene glycol,
c) about 5 mg/mL to about 50 mg/mL of cyclodextrin selected from the group consisting of sulfobutylether β-cyclodextrin (SBECD) or hydroxypropyl β-cyclodextrin.
d) a co-solvent, and
e) hydrochloric acid.
   wherein the composition has a pH between 2.0 to 6.0.

In another embodiment, the present invention relates to a liquid pharmaceutical composition comprising:
a) 0.1 mg/mL to 1 mg/mL of lurbinectedin or a pharmaceutically acceptable salt thereof,
b) about 200 mg/mL to about 300 mg/mL of polyethylene glycol,
c) about 10 mg/mL to about 30 mg/mL of sulfobutylether-β-cyclodextrin,
d) a co-solvent selected from the group consisting of benzyl alcohol or ethanol, and
e) hydrochloric acid, and
   wherein the composition has a pH between 2.0 to 6.0.

In another embodiment, the present invention relates to a liquid pharmaceutical composition comprising:
a) 0.5 mg/mL to 1 mg/mL of lurbinectedin or a pharmaceutically acceptable salt thereof,
b) about 200 mg/mL to about 250 mg/mL of polyethylene glycol,
c) about 15 mg/mL to about 25 mg/mL of sulfobutylether-β-cyclodextrin,
d) a co-solvent selected from the group consisting of benzyl alcohol or ethanol, and
e) hydrochloric acid,
   wherein the composition has a pH between 2.0 to 6.0.

In another embodiment, the present invention relates to a liquid pharmaceutical composition comprising:
a) 0.5 mg/mL of lurbinectedin or a pharmaceutically acceptable salt thereof,
b) about 200 mg/mL to about 250 mg/mL of polyethylene glycol,
c) about 15 mg/mL to about 25 mg/mL of sulfobutylether-β-cyclodextrin,
d) a co-solvent selected from the group consisting of benzyl alcohol or ethanol, and
e) hydrochloric acid,
   wherein the composition has a pH between 2.0 to 6.0.

In another embodiment, the present invention relates to a liquid pharmaceutical composition comprising:
a) 1 mg/mL of lurbinectedin or a pharmaceutically acceptable salt thereof,
b) about 200 mg/mL to about 250 mg/mL of polyethylene glycol,
c) about 15 mg/mL to about 25 mg/mL of sulfobutylether-β-cyclodextrin,
d) a co-solvent selected from the group consisting of benzyl alcohol or ethanol, and
e) hydrochloric acid,
   wherein the composition has a pH between 2.0 to 6.0.

In one embodiment, the present invention relates to a liquid pharmaceutical composition, wherein the composition is free of carboxylic acid.

In another embodiment, the present invention relates to a liquid pharmaceutical composition, wherein the composition is free of monosaccharide.

In another embodiment, the present invention relates to a liquid pharmaceutical composition, wherein the composition is free of disaccharide.

In one embodiment, the present invention relates to a liquid pharmaceutical composition, wherein the composition is free of carboxylic acid, is free of monosaccharide and is free of disaccharide.

In another embodiment, the present invention relates to a liquid pharmaceutical composition, wherein the composition is not a lyophilized product.

In another embodiment, the present invention relates to a ready-to-dilute liquid pharmaceutical composition comprising:
a) 0.1 mg/mL to 1 mg/mL of lurbinectedin or a pharmaceutically acceptable salt thereof,
b) about 200 mg/mL to about 300 mg/mL of polyethylene glycol,
c) about 10 mg/mL to about 30 mg/mL of sulfobutylether-β-cyclodextrin,
d) a co-solvent selected from the group consisting of benzyl alcohol or ethanol, and
e) hydrochloric acid,
   wherein the composition has a pH between 2.0 to 6.0, is free of carboxylic acid, is free of monosaccharide and is free of disaccharide.

In another embodiment, the present invention relates to a ready-to-dilute liquid pharmaceutical composition comprising:
a) 0.5 mg/mL to 1 mg/mL of lurbinectedin or a pharmaceutically acceptable salt thereof,
b) about 200 mg/mL to about 250 mg/mL of polyethylene glycol,
c) about 15 mg/mL to about 25 mg/mL of sulfobutylether-β-cyclodextrin,
d) a co-solvent selected from the group consisting of benzyl alcohol or ethanol, and
e) hydrochloric acid,
   wherein the composition has a pH between 2.0 to 6.0, is free of carboxylic acid, is free of monosaccharide and is free of disaccharide.

In another embodiment, the present invention relates to a ready-to-dilute liquid pharmaceutical composition comprising:
a) 0.5 mg/mL of lurbinectedin or a pharmaceutically acceptable salt thereof,
b) about 200 mg/mL to about 250 mg/mL of polyethylene glycol,
c) about 15 mg/mL to about 25 mg/mL of sulfobutylether-β-cyclodextrin,
d) a co-solvent selected from the group consisting of benzyl alcohol or ethanol, and
e) hydrochloric acid,
   wherein the composition has a pH between 2.0 to 6.0, is free of carboxylic acid, is free of monosaccharide and is free of disaccharide.

In another embodiment, the present invention relates to a ready-to-dilute liquid pharmaceutical composition comprising:
a) 1 mg/mL of lurbinectedin or a pharmaceutically acceptable salt thereof,
b) about 200 mg/mL to about 250 mg/mL of polyethylene glycol,
c) about 15 mg/mL to about 25 mg/mL of sulfobutylether-β-cyclodextrin,
d) a co-solvent selected from the group consisting of benzyl alcohol or ethanol, and
e) hydrochloric acid,
   wherein the composition has a pH between 2.0 to 6.0, is free of carboxylic acid, is free of monosaccharide and is free of disaccharide.

The liquid pharmaceutical compositions of the present invention do not exhibit substantial Lurbinectedin degradation when exposed to certain conditions. Stability may be measured by any suitable method, e.g., by high-performance liquid chromatography (HPLC). In a particular embodiment, the liquid pharmaceutical compositions of the present invention do not exhibit substantial Lurbinectedin degradation under photolytic conditions, thermal conditions or combinations thereof. In another particular embodiment, the liquid pharmaceutical compositions of the present invention do not exhibit substantial Lurbinectedin degradation under forced stability conditions, accelerated stability conditions, real-time stability conditions or a combination thereof.

In some embodiments, the liquid pharmaceutical compositions disclosed herein do not exhibit substantial degradation under accelerated conditions at 25 C/60% RH for at least 3 months, at least 4 months, at least 6 months, or at least 6 months.

In one embodiment, the liquid pharmaceutical composition disclosed herein in exhibits about 5% or less degradation of Lurbinectedin over at least a three month period when exposed to 2-8° C. such as, for example, about 4.5% or less, 4.0% or less, about 3.5% or less, about 3.0% or less, about 2.5% or less, about 2.0% or less, about 1.5% or less or about 1.0% or less.

In some embodiments, the liquid pharmaceutical composition disclosed herein do not exhibit substantial degradation under refrigerated temperature conditions (e.g. 2-8° C.) for at least 3 months, at least 6 months, at least 8 months, at least 10 months, at least 12 months, or at least 24 months.

In some embodiments, the liquid pharmaceutical compositions exhibits about 5% or less degradation of the compound of Formula (I) over at least a three month period when exposed to 2-8 C, such as, for example, about 4.5% or less, 4.0% or less, about 3.5% or less, about 3.0% or less, about 2.5% or less, about 2.0% or less, about 1.5% or less or about 1.0% or less.

Methods of measuring Lurbinectedin degradation are known to a person of skill in the art, e.g., monitoring the amount of Lurbinectedin in the liquid pharmaceutical composition by HPLC over a period of time. An exemplary method is provided below.

The chromatographic analysis was performed using a Kinetex Biphenyl 100 Å 250×4.6 mm, 5.0 μm column (Phenomenex, Part No: 00G-4627-E0) in conjunction with a Ghost Buster Column (4.6×50 mm, Welch, Part No: 06100-31000). The Ghost Buster Column was installed between the gradient mixer and the auto-sampler. The flow rate was set at 0.8 mL/min, with a detection wavelength of 210 nm and an injection volume of 10 μL. The column temperature was maintained at 40° C., and the auto-sampler temperature at 5° C. The total runtime for the analysis was 35 minutes. Methanol, chilled at 2-8° C., was used as the diluent, and the needle wash was performed using methanol. The elution was carried out using a gradient method.

For buffer preparation, 3.0 mL of perchloric acid (70%) was transferred into a 1000 mL beaker containing ultrapure water and mixed well. Mobile phase-A consisted of 100% buffer, while Mobile phase-B was a mixture of acetonitrile and buffer in a 750:250 (v/v) ratio. The gradient program was as follows

| Time (min) | Mobile phase A (%) | Mobile phaseB (%) |
|---|---|---|
| 0 | 55 | 45 |
| 2 | 55 | 45 |
| 18 | 30 | 70 |
| 18.5 | 10 | 90 |
| 26 | 10 | 90 |
| 26.5 | 55 | 45 |
| 35 | 55 | 45 |

standard solution: (0.1 mg/mL)-2.5 mg of Lurbinectedin reference/working standard into 25 mL volumetric flask with diluent.
test sample preparation: (0.1 mg/mL)-2.0 mL of test sample into 20 mL of volumetric flask and dilute to volume with diluent.

### Injection Sequence as Below.

| S. No | Name of the solution | No. of Injections |
|---|---|---|
| 1. | Blank (Diluent) | 2 |
| 2. | Standard Solution | 5 |
| 3. | Test solution | 1 |
| 4. | Standard Solution as Bracketing | 1 |

### Calculation:

Calculate the % w/w of each impurity by following formula:

$$\% Assay = \frac{Ai \times Sw \times 20 \times P}{As \times 25 \times Sv \times LC}$$

Where,
Ai=Area of Lurbinectedin in sample solution.
As=Average Area of Lurbinectedin in standard solution
Sw=Weight of Lurbinectedin working/Reference standard taken in mg.
P=Potency of Lurbinectedin working/Reference Standard.
Sv=Volume of test sample taken in mL.
LC=Label claim (1 mg/mL)

In another embodiment this invention discloses a process to prepare the ready-to-dilute stable parenteral composition which comprises of:
a) adding required quantity of water in a manufacturing vessel and add cyclodextrin to it and stir to obtain a clear solution,
b) adjusting the pH using suitable acidifying agent,
c) to the above vessel add lurbinectedin and stir to obtain a clear solution,
d) to the above vessel add polyethylene glycol and stir to obtain a clear solution, and
e) filtering the solution, filling the filtered solution into vials; stoppering and sealing the vials and storing the vials in suitable shipper.

In another embodiment this invention discloses a process to prepare the ready-to-dilute stable parenteral composition which comprises of:
a) adding required quantity of water in a manufacturing vessel and add cyclodextrin to it and stir to obtain a clear solution,
b) to the above vessel solvents benzyl alcohol and ethanol be added
c) adjusting the pH using suitable acidifying agent,
d) to the above vessel add lurbinectedin and stir to obtain a clear solution,
e) to the above vessel add polyethylene glycol and stir to obtain a clear solution, and
f) filtering the solution, filling the filtered solution into vials; stoppering and sealing the vials and storing the vials in suitable shipper.

In embodiment this invention discloses a process to prepare the ready-to-dilute stable parenteral composition which comprises of:
a) adding required quantity of water in a manufacturing vessel and add sulfobutylether-β-cyclodextrin to it and stir to obtain a clear solution,
b) adjusting the pH using suitable acidifying agent to 2.5,
c) to the above vessel add lurbinectedin and stir to obtain a clear solution,
d) to the above vessel add polyethylene glycol and stir to obtain a clear solution, and
e) filtering the solution, filling the filtered solution into vials; stoppering and sealing the vials and storing the vials in suitable shipper.

Another embodiment of the present invention relates to liquid injectable compositions comprising about 0.01 mg/mL to about 1 mg/mL lurbinectedin or its pharmaceutically acceptable salts thereof; about 200 mg/mL to about 400 mg/mL polyethylene glycol and about 15 mg/mL to about 35 mg/mL sulfobutylether-β-cyclodextrin, contains no more than 2% total impurities, when stored at least one month, or at least 3 months, or at least five months at a temperature from 2-8° C.

In the above-mentioned embodiment, the combination of PEG and cyclodextrin stabilizes the composition and decreases the total impurity levels to less than 2% when stored at 2-8° C. for at least one month, or at least 3 months, or at least five months.

Another embodiment of the present invention relates to liquid injectable compositions comprising about 0.01 mg/mL to about 1 mg/mL lurbinectedin or its pharmaceutically acceptable salts thereof; about 200 mg/mL to about 400 mg/mL polyethylene glycol and about 15 mg/mL to about 35 mg/mL sulfobutylether-β-cyclodextrin, contains no more than 1% total impurities, when stored for at least one month, or at least 3 months, or at least five months at a temperature from 2-8° C.

Another embodiment of the present invention relates to liquid injectable compositions comprising about 0.01 mg/mL to about 1 mg/mL lurbinectedin or its pharmaceutically acceptable salts thereof; about 200 mg/mL to about 400 mg/mL polyethylene glycol and about 15 mg/mL to about 35 mg/mL sulfobutylether-β-cyclodextrin, contains no more than 0.5% deacetylated lurbinectedin, when stored for at least one month, or at least 3 months, or at least five months at a temperature from 2-8° C.

Another embodiment of the present invention relates to liquid injectable compositions comprising about 0.01 mg/mL to about 1 mg/mL lurbinectedin or its pharmaceutically acceptable salts thereof; about 200 mg/mL to about 400 mg/mL polyethylene glycol and about 15 mg/mL to about 35 mg/mL sulfobutylether-β-cyclodextrin, contains no more than 0.1% deacetylated lurbinectedin, when stored for at least one month, or at least 3 months, or at least five months at a temperature from 2-8° C.

Injectable formulations may take any route including intramuscular, intra-peritoneal, intravenous or subcutaneous administration. Preferred are subcutaneous or intravenous route of administration for the composition of the present invention.

According to another embodiment, the present invention relates to the liquid injectable composition comprising lurbinectedin; wherein the injectable composition is packaged in a container suitable for both single and multi-use. The preferred containers include an ampoule, a vial, a pre-filled syringe, and intravenous bag.

According to another embodiment, the present invention relates to liquid injectable composition comprising lurbinectedin; wherein the injectable composition is packaged in a container suitable for multi-use, wherein the injectable composition comprises solvent system suitable for injection, solubilizer, stabilizer, buffering agent, antioxidants, and preservative, wherein the liquid injectable compositions are ready-to-dilute.

According to another embodiment, the present invention relates to liquid injectable composition comprising lurbinectedin; wherein the injectable composition is packaged in a container suitable for multi-use, wherein the injectable composition comprises solvent system suitable for injection, solubilizer, stabilizer, buffering agent, antioxidants, and preservative, wherein the liquid injectable compositions are ready-to-use.

The following examples further describe certain specific formulations of the invention and demonstrate the practice and advantages thereof. A general manufacturing process is given for Examples 1-4. It is to be understood that the examples and manufacturing are given by way of illustration only and are not intended to limit the scope of the invention in any manner.

### EXAMPLES

### Example-1

### Manufacturing Formula:

TABLE 1

| S. No. | Formulation | Mg/mL |
|---|---|---|
| 1 | Lurbinectedin | 1.0 |
| 2 | Sulfobutylether-β-cyclodextrin | 20 |
| 3 | Benzyl alcohol | 20.0 |
| 4 | Ethanol | 100.0 |
| 5 | polyethylene glycol 400 | 367 (0.3 ml) |
| 6 | Hydrochloric acid | q.s. to pH 2.5 |
| 7 | Water for injection | q.s. to mL |

### Manufacturing Process

The required quantity of Sulfobutylether-β-cyclodextrin and Water for injection were weighed to the manufacturing vessel and stirred for giving a clear solution. Ethanol and Benzyl alcohol were added to this and stirred. pH of this solution adjusted to 2.5 using 1N hydrochloric acid. The required quantity of API was added to this solution and stirred. polyethylene glycol 400 was added and mixed thoroughly to obtain a clear solution. Water for injection was added to make up the volume.

### Stability Data:

Formulation 01

| | Condition | | | | | | |
|---|---|---|---|---|---|---|---|
| | 2-8° C. | | | | 25° C./60% RH | | |
| | Initial | 1M | 3M | 5M | 1M | 3M | 5M |
| pH | 4.16 | 4.06 | 4.06 | 4.16 | 3.97 | 4.02 | 4.19 |
| Assay | 109.7 | 106.3 | 98.2 | 106.1 | 105.5 | 97.9 | 104.7 |
| RRT | Related substances % W/V as determined by HPLC | | | | | | |
| Des Acetyl Impurity | BPQL | 0.04 | 0.04 | 0.05 | BPQL | 0.05 | 0.17 |
| Des Hydroxy Impurity | 0.05 | 0.06 | 0.09 | 0.04 | 0.07 | 0.06 | 0.04 |
| Mom Impurity | BPQL | ND | BPQL | BPQL | BDL | BPQL | BPQL |
| Cyno des mom Impurity | 0.05 | 0.06 | 0.07 | 0.07 | 0.08 | 0.09 | 0.09 |
| Highest Unknown | 0.13 | 0.19 | 0.26 | 0.30 | 0.46 | 0.35 | 0.17 |
| TOTAL | 0.86 | 0.97 | 0.93 | 1.11 | 1.46 | 1.50 | 1.6 |

BPQL—below quantification limit
BDL—below detection limit

### Example-2

### Manufacturing Formula:

TABLE 2

| S. No. | Formulation | Mg/mL |
|---|---|---|
| 1 | Lurbinectedin | 1.0 |
| 2 | Sulfobutylether-β-cyclodextrin | 20 |
| 5 | polyethylene glycol 400 | 225 mg (0.2 ml) |
| 6 | Hydrochloric acid | q.s. to pH 2.5 |
| 7 | Water for injection | q.s. to mL |

### Manufacturing Process

The required quantity of Sulfobutylether-β-cyclodextrin and Water for injection were weighed to the manufacturing vessel and stirred for giving a clear solution. pH of this solution adjusted to 2.5 using 1N hydrochloric acid. The required quantity of API was added to this solution and stirred. PEG was added and mixed thoroughly to obtain a clear solution. Water for injection was added to make up the volume.

### Stability Data:

Formulation 02

| | Condition | | | | | | |
|---|---|---|---|---|---|---|---|
| | 2-8° C. | | | | 25° C./60% RH | | |
| | Initial | 1M | 3M | 5M | 1M | 3M | 5M |
| pH | 4.34 | 4.27 | 4.23 | 4.33 | 4.21 | 4.28 | 4.30 |
| Assay | 101.5 | 101.4 | 98.8 | 102.7 | 99.4 | 97.0 | 99.6 |
| RRT | Related substances % W/V | | | | | | |
| Des Acetyl Impurity | BPQL | BPQL | BPQL | 0.05 | BPQL | BPQL | 0.05 |
| Des Hydroxy Impurity | 0.04 | 0.05 | 0.04 | 0.04 | 0.04 | BPQL | 0.04 |
| Mom Impurity | BPQL | BPQL | BPQL | BPQL | BPQL | BPQL | BPQL |
| Cyno des mom Impurity | 0.06 | 0.07 | 0.07 | 0.07 | 0.08 | 0.08 | 0.08 |
| Highest Unknown | 0.11 | 0.71 | 0.12 | 0.10 | 0.11 | 0.12 | 0.2 |
| TOTAL | 0.47 | 0.71 | 0.59 | 0.72 | 1.19 | 1.33 | 1.62 |

### Example-3

### Manufacturing Formula:

TABLE 3

| S. No. | Formulation | Mg/mL |
|---|---|---|
| 1 | Lurbinectedin | 0.5 |
| 2 | Hydrochloric acid | q.s. to pH 2.5 |
| 3 | Water for injection | q.s. to mL |

### Manufacturing Process

The required quantity of Water for injection was weighed to the manufacturing vessel and pH of this solution adjusted to 2.5 using 1N hydrochloric acid. The required quantity of API was added to this solution and stirred to obtain a clear solution. Water for injection was added to make up the volume.

### Stability Data:

Formulation 03

| | Condition | | | | | | |
|---|---|---|---|---|---|---|---|
| | 2-8° C. | | | | 25° C./60% RH | | |
| | Initial | 1M | 3M | 5M | 1M | 3M | 5M |
| pH | 2.59 | 2.52 | 2.53 | 2.57 | 2.53 | 2.53 | 2.62 |
| Assay | 104.7 | 103.9 | 100.2 | 104.9 | 101.5 | 96.4 | 98.1 |
| RRT | Related substances % W/V | | | | | | |
| Des Acetyl Impurity | 0.08 | 0.18 | 0.27 | 0.34 | 0.21 | 0.19 | 0.17 |
| Des Hydroxy Impurity | 0.04 | 0.06 | 0.07 | 0.04 | 0.07 | 0.03 | 0.03 |
| Mom Impurity | ND | BDL | BPQL | BPQL | ND | ND | ND |
| Cyno des mom Impurity | 0.05 | 0.06 | 0.06 | 0.06 | 0.06 | 0.06 | 0.05 |
| Highest Unknown | 0.11 | 0.11 | 0.10 | 0.12 | 0.13 | 0.38 | 0.65 |
| TOTAL | 0.45 | 0.82 | 0.86 | 0.09 | 1.42 | 2.90 | 4.58 |

BPQL—below quantification limit
BDL—below detection limit
ND—Not detected

### Example-4

### Manufacturing Formula:

TABLE 4

| S. No. | Formulation | Mg/mL |
|---|---|---|
| 1 | Lurbinectedin | 0.5 |
| 2 | sulphuric acid | q.s. to pH 2.5 |
| 3 | Water for injection | q.s. to mL |

### Manufacturing Process

The required quantity of Water for injection was weighed to the manufacturing vessel and pH of this solution adjusted to 2.5 using sulphuric acid. The required quantity of API was added to this solution and stirred to obtain a clear solution. Water for injection was added to make up the volume.

### Stability Data:

Formulation 04

| | Condition | | | | | | |
|---|---|---|---|---|---|---|---|
| | 2-8° C. | | | | 25° C./60% RH | | |
| | Initial | 1M | 3M | 5M | 1M | 3M | 5M |
| pH | 2.65 | 2.61 | 2.62 | 2.70 | 2.68 | 2.63 | 2.68 |
| Assay | 102.6 | 102.5 | 99.59 | 103.8 | 101.8 | 94.66 | 95.37 |
| RRT | Related substances % W/V | | | | | | |
| Des Acetyl Impurity | 0.04 | 0.15 | 0.25 | 0.39 | 0.20 | 0.11 | 0.12 |
| Des Hydroxy Impurity | 0.04 | 0.05 | 0.06 | 0.04 | 0.05 | 0.04 | 0.03 |
| Mom Impurity | ND | ND | BPQL | BPQL | ND | BPQL | BPQL |
| Cyno des mom Impurity | 0.05 | 0.05 | 0.06 | 0.06 | 0.06 | 0.05 | 0.05 |
| Highest Unknown | 0.12 | 0.11 | 0.10 | 0.08 | 0.15 | 0.61 | 0.70 |
| TOTAL | 0.50 | 0.94 | 0.95 | 1.34 | 1.68 | 4.17 | 4.89 |

BDL—below detection limit
ND—Not detected.

We claim:

1. A liquid pharmaceutical composition comprising:
a) 0.1 mg/mL to 2 mg/mL of lurbinectedin or a pharmaceutically acceptable salt thereof,
b) about 100 mg/mL to about 500 mg/mL of polyethylene glycol, and
c) about 5 mg/mL to about 50 mg/mL of cyclodextrin,
wherein the composition has a pH between 2.0 to 6.0.

2. The pharmaceutical composition according to claim 1, wherein the composition is a ready-to-dilute composition.

3. The pharmaceutical composition according to claim 1, wherein lurbinectedin in the composition has a concentration of about 0.5 mg/mL or about 1 mg/mL.

4. The pharmaceutical composition according to claim 1, wherein the polyethylene glycol is selected from PEG 200 or PEG 300 or PEG 400 or PEG 600 or PEG 900.

5. The pharmaceutical composition according to claim 1, wherein the cyclodextrin is sulfobutylether-β-cyclodextrin.

6. The pharmaceutical composition according to claim 1, further comprising a co-solvent selected from ethanol, benzyl alcohol, butanediol, isopropanol, alkyl glycols for example, ethylene glycol, propylene glycol, and butylene glycol, glycerol, polysorbate or mixtures thereof.

7. The pharmaceutical composition according to claim 6, wherein the co-solvent is ethanol.

8. The pharmaceutical composition according to claim 6, wherein the co-solvent is benzyl alcohol.

9. The pharmaceutical composition according to claim 1, wherein the composition further comprises an inorganic acid.

10. The pharmaceutical composition according to claim 9, wherein the inorganic acid is hydrochloric acid.

11. The pharmaceutical composition according to claim 1, wherein the composition has a pH between 2.0-4.0.

12. The pharmaceutical composition according to claim 1, wherein the composition has a pH of about 4.0.

13. The pharmaceutical composition according to claim 1, wherein the composition is free of disaccharides.

14. The pharmaceutical composition according to claim 1, wherein the composition is free of monosaccharides.

15. The pharmaceutical composition according to claim 1, wherein the composition is free of carboxylic acids.

16. The pharmaceutical composition according to claim 1, wherein the composition is free of disaccharides, is free of monosaccharides, and is free of carboxylic acids.

17. The pharmaceutical composition according to claim 1, wherein the composition is stable at 2-8 C for a period of at least 3 months.

18. The pharmaceutical composition according to claim 1, wherein the composition is stable at 2-8 C for a period of at least 6 months.

19. The pharmaceutical composition according to claim 1, wherein the composition is stable at stable 2-8 C for a period of at least 12 months.

20. The pharmaceutical composition according to claim 1, wherein the composition is stable at 2-8 C for a period of at least 24 months.

21. The pharmaceutical composition according to claim 1, wherein the composition maintains at least 98% of Lurbinectedin content and/or contains less than 2% of total impurities as determined by HPLC, after storage at 2-8° C. for a period of at least 6 months.

22. The pharmaceutical composition according to claim 1, wherein the composition maintains less than 2% of impurities as determined by HPLC, after storage at 2-8° C. for a period of at least 6 months.

23. The pharmaceutical composition according to claim 1, wherein the composition maintains less than 0.5% deacetyl impurity as determined by HPLC, after storage at 2-8° C. for a period of at least 6 months.

24. A ready-to-dilute liquid pharmaceutical composition comprising:
a) 0.01 mg/mL to 1.0 mg/mL of lurbinectedin or a pharmaceutically acceptable salt thereof,
b) about 100 mg/mL to about 500 mg/mL of polyethylene glycol,
c) about 5 mg/mL to about 50 mg/mL of sulfobutylether-β-cyclodextrin, and
d) hydrochloric acid,
wherein the composition has a pH between 2.0 to 6.0.

25. The pharmaceutical composition according to claim 24, wherein the composition consists of:
a) 0.5 mg/mL to 1.0 mg/mL of lurbinectedin or a pharmaceutically acceptable salt thereof,
b) about 100 mg/mL to about 500 mg/mL of polyethylene glycol,
c) about 5 mg/mL to about 50 mg/mL of sulfobutylether-β-cyclodextrin, and
d) about 0.2 mg/mL to about 0.8 mg/mL hydrochloric acid,
wherein the composition has a pH between 2.0 to 6.0.

26. The pharmaceutical composition according to claim 24, wherein the composition consists of:
a) 0.5 mg/mL to 1.0 mg/mL of lurbinectedin or a pharmaceutically acceptable salt thereof,
b) about 100 mg/mL to about 500 mg/mL of polyethylene glycol,
c) about 5 mg/mL to about 50 mg/mL of sulfobutylether-β-cyclodextrin,
d) about 0.2 mg/mL to about 0.8 mg/mL hydrochloric acid, and
e) about 50 mg/mL to about 200 mg/mL ethanol.
wherein the composition has a pH between 2.0 to 6.0.

27. The pharmaceutical composition according to claim 24, wherein the composition consists of:
a) 0.01 mg/mL to 1 mg/mL of lurbinectedin or a pharmaceutically acceptable salt thereof,
b) about 100 mg/mL to about 500 mg/mL of polyethylene glycol,
c) about 5 mg/mL to about 50 mg/mL of sulfobutylether-β-cyclodextrin,
d) about 0.2 mg/mL to about 0.8 mg/mL hydrochloric acid, and
e) about 5 mg/mL to about 40 mg/mL benzyl alcohol.
wherein the composition has a pH between 2.0 to 6.0.

28. The pharmaceutical composition according to claim 24, wherein the composition consists of:
a) 0.01 mg/mL to 1 mg/mL of lurbinectedin or a pharmaceutically acceptable salt thereof,
b) about 100 mg/mL to about 500 mg/mL of polyethylene glycol,
c) about 5 mg/mL to about 50 mg/mL of sulfobutylether-β-cyclodextrin,
d) about 0.2 mg/mL to about 0.8 mg/mL hydrochloric acid,
e) about 50 mg/mL to about 200 mg/mL ethanol, and
f) about 5 mg/mL to about 40 mg/mL benzyl alcohol,
wherein the composition has a pH between 2.0 to 6.0.

29. The pharmaceutical composition according to claim 24, wherein the composition consists of:
a) 0.01 mg/mL to 1 mg/mL of lurbinectedin or a pharmaceutically acceptable salt thereof,
b) about 100 mg/mL to about 500 mg/mL of polyethylene glycol,
c) about 5 mg/mL to about 50 mg/mL of sulfobutylether-β-cyclodextrin, and
d) about 0.2 mg/mL to about 0.8 mg/mL hydrochloric acid,
wherein the composition has a pH between 2.0 to 6.0, is free of monosaccharides, disaccharides and/or is free of organic carboxylic acid.
